(19) United States
(12) Patent Application Publication
(10) Pub. No.: US 2021/0322084 A1
(43) Pub. Date: Oct. 21, 2021

(54) COLD SOLUTION FOR FAT REDUCTION

(71) Applicant: MIRAKI INNOVATION THINK TANK LLC, Cambridge, MA (US)

(72) Inventors: Christopher VELIS, Lexington, MA (US); Karen MILLER, South Dartmouth, MA (US); Tarik S. CHAUDHRY, Boston, MA (US)

(73) Assignee: MIRAKI INNOVATION THINK TANK LLC, Cambridge, MA (US)

(21) Appl. No.: 17/283,698

(22) PCT Filed: Oct. 10, 2019
(86) PCT No.: PCT/US2019/055605

§ 371 Date: Apr. 8, 2021

Related U.S. Application Data
(60) Provisional application No. 62/745,054, filed on Oct. 12, 2018.

(51) Int. Cl. A61B 18/02; A61K 9/00; A61K 9/08; A61K 47/02; A61K 47/26

(52) U.S. Cl. CPC A61B 18/0218; A61K 9/0019; A61K 9/08; A61B 2018/00464; A61K 47/26; A61B 2018/0293; A61K 47/02

(57) ABSTRACT

The present invention is a cold solution for reducing adipose tissue or fat. Delivering the cold solution to a region of lipid-rich tissue induces the natural cell death of adipocytes and thereby reduces the tissue. Following cell death, localized panniculitis or inflammation of the tissue leads to further reduction as adipocytes are removed as a result of phagocytosis. The cold solution does not have ice particles. One or more additives, including salt, sugar, thickeners, freezing point depressants, surfactants, and excipients can be added to the cold solution to facilitate/enhance fat reduction. Fat reduction by cold solution injection can be used to improve one's appearance or to treat a weight-related disorder, such as obesity.

### TECHNICAL FIELD

This disclosure relates to cold solution for fat reduction.

### BACKGROUND

The demand for procedures to reduce fat, often referred to as body contouring procedures, is large and continues to rise, especially with the increasing number of minimally and non-invasive therapies available. According to the American Society of Aesthetic Plastic Surgery (ASAPS), in 2014, consumers spent approximately $12 billion on aesthetic procedures, including invasive, minimally invasive, and non-invasive fat reduction procedures.

Invasive fat reduction procedures on the market include liposuction, abdominoplasty (“tummy tuck”), gluteoplasty (buttock lifts), brachioplasty (arm lift), thighplasty (thigh lift), lower rhytidectomy (neck lift), and mentoplasty (chin tightening). Invasive therapies carry risks associated with surgical procedures, some of which can be life threatening. These include infection, scarring, perforation of organs and vessels, and hemorrhage. Additionally, invasive therapies are often painful and typically require a lengthy recovery period.

Minimally invasive fat reduction procedures include laser-assisted liposuction, laser lipolysis (e.g., the breakdown of lipids), radio frequency lipolysis, ultrasound lipolysis, and injection lipolysis (e.g. injection of deoxycholic acid; KYBELLA). These procedures may require a surgical incision and/or the delivery of chemicals into the body, which can carry risks to the patient, and are often painful and produce non-uniform results.

Noninvasive procedures currently on the market include the use of radio frequency, lasers, and ultrasound, as well as the application of cold temperatures to the surface of the skin (e.g. CoolSculpting by Zeltiq Aesthetics, Inc.). These therapies are often time consuming and painful, while delivering minimal results.

Recently, minimally and non-invasive procedures to delivery cold topically to fat tissue have been developed, with a non-invasive therapy known as CoolSculpting, as noted above, currently on the market. These procedures are based on the principle that fat cells (adipose tissue) are more sensitive to cold temperatures than the skin or other surrounding tissues, with the cold temperatures causing the fat cells to undergo apoptosis, a natural biological process through which fat cells are eliminated from the body. Non-invasive delivery of cool temperatures directly to the skin, however, can be painful, may produce unsatisfactory results, and is very time consuming, with the associated apparatus needing to be held on a patient's skin for a lengthy amount of time.

### SUMMARY

Excess fat poses a host of local and systemic problems, including increased risk for cardiovascular disease, type II diabetes, and cancer, associated particularly with excess visceral fat, and secondary problems due to being overweight including musculoskeletal problems, arthritis, and difficulty exercising. Based on the premise that fat cells are more easily damaged by cooling than skin cells, cryolipolysis was developed as a nonsurgical way to destroy fat cells. Cold is applied to a region of lipid-rich tissue (fat), effectively crystallizing adipose cells and inducing apoptosis, a natural cell death. Furthermore, localized panniculitis or inflammation of the tissue occurs later on leading to further removal of adipocytes (fat cells) as a result of phagocytosis.

The present invention provides a cold solution for cooling adipose tissue to induce apoptosis which, in turn, reduces the adipose tissue. The cold solution can be injected through a subject's skin, noninvasively, using a syringe or cannula.

The cold solution can include pure water. In some embodiments, the cold solution can include mixtures of water and one or more additives that facilitate and/or enhance the reduction of adipose tissue. These additives include salt, freezing point depressants, surfactants, and excipients. The cold solution can be cooled or supercooled to a temperature just before spontaneous nucleation occurs. In some embodiments, the cold solution is cooled or supercooled to a temperature approximate to or lower than where spontaneous nucleation occurs, then warmed such that all ice particles melt prior to delivery to a subject. The cold solution can be delivered to adipose tissue located in any number of places on the human body, such as around the flank, abdomen, thigh area, upper arm, and submental area under the chin. Fat reduction by cold solution can be used as part of a treatment for obesity or other weight-related disorder. Such a treatment can include selecting a subject to whom to administer cold solution, creating a treatment plan for the subject, administrating an effective amount of cold solution to treat the condition, and assessing the result of administrating cold solution.

### BRIEF DESCRIPTION OF DRAWINGS

FIG. 1A is a diagram of subcutaneous fat locations in the body.

FIG. 1B is a diagram of subcutaneous and visceral fat locations within the abdominal area.

FIG. 1C is a diagram of brown adipose tissue locations in the body.

FIG. 2 is a view of an example device for delivering a cold solution to adipose tissue.

FIG. 3 is a diagram a cold solution being delivered to subcutaneous adipose tissue.

FIG. 4 is a view of an example device for delivering a cold solution to adipose tissue.

### DETAILED DESCRIPTION

The present invention provides a cold solution for cooling adipose tissue to induce apoptosis which, in turn, reduces the adipose tissue. Based on the premise that fat cells are more easily damaged by cooling than skin cells, cryolipolysis can be applied as a nonsurgical way to destroy fat cells. For example, cold is applied to a region of lipid-rich tissue (such as fat), effectively crystallizing adipose cells and inducing apoptosis, a natural cell death. Furthermore, localized panniculitis or inflammation of the tissue occurs later on leading to further removal of adipocytes (fat cells) as a result of phagocytosis.

The cold solution can be cooled or supercooled such that it does not have ice particles and can be injected through a subject's skin to adipose tissue underneath, noninvasively, using a syringe or cannula. In some embodiments, the cold solution comprises water. In some embodiments, the cold solution comprises water and one or more additives.

In some embodiments, the cold solution is cooled or supercooled to a temperature just before spontaneous nucleation occurs. In some embodiments, the cold solution is cooled or supercooled to a temperature approximate to or lower than where spontaneous nucleation occurs, then warmed such that all ice particles melt prior to delivery to a subject.

One example of a cold solution is water that is supercooled. Water normally freezes at 273.15 K (0° C. or 32° F.), but it can be supercooled at standard pressure down to its crystal homogeneous nucleation at almost 224.8 K (−48.3° C./−55° F.). The supercooling process requires that water be pure and free of nucleation sites. This can be done by processes like reverse osmosis or chemical demineralization. Rapidly cooling water at a rate on the order of 10^6 K/s avoids crystal nucleation and water becomes a glass, i.e., an amorphous (non-crystalline) solid. The temperature of the cold solution can be cooled to temperature ranging from at about 10° C. to at about −50° C.

In one embodiment, the additives comprise one or more freezing point depressants lowering the freezing point of the cold solution. Exemplary freezing point depressants include salts (e.g. sodium chloride, potassium, calcium, magnesium, hydrogen phosphate, hydrogen or carbonate), ions, Lactated Ringer's solution, sugars (e.g., glucose, sorbitol, mannitol, or hetastarch, sucrose), biocompatible surfactants such as glycerol, other polyols, other sugar alcohols, and/or urea, and the like. In one aspect, the freezing point depressant content of the cold solution is between about 0.5% and about 40%, between about 10% and about 30%, or between about 12% and about 22%. In some embodiments, the cold solution includes a biocompatible surfactant such as glycerol. These ingredients can also serve as a cryo-protectant for non-lipid-rich cells. In some embodiments, the additives comprise at least one thickener or an additive affecting the viscosity of the solution, for example, sodium carboxymethylcellulose (CMC) or xanthan gum.

In order to produce a cold solution that selectively destructs lipid-rich cells while avoiding acute unselective necrosis, the cold solution can be isotonic relative to the subject's cells, e.g., having an osmolarity of about 308 mOsm/L. An exemplary cold solution-composition includes normal saline and 2% glycerol. In non-selective, broader destructive slurries, colder temperatures and greater destructive power can be achieved by increasing the solute concentration (e.g., to 20% w/v saline) to form a hypertonic solution (i.e., a solution having an osmolarity greater than about 308 mOsm/L) that will also disrupt cells through osmotic pressure. It is also contemplated that the cold solutions further comprise a therapeutic compound.

The cold solution can comprise additional excipients, such as those found in Sougata Pramanick et al., “Excipient Selection In Parenteral Formulation Development,” 45(3) Pharma Times 65-77 (2013), which is incorporated herein by reference. Exemplary excipients include bulking agents, such as sucrose, lactose, trehalose, mannitol, sorbitol, glucose, raffinose, glycine, histidine, PVP (K40); buffering agents, such as sodium citrate, sodium phosphate, sodium hydroxide, tris base-65, tris acetate, tris HCl-65; tonicity modifiers, such as dextrose; collapse temperature modifiers such as dextran, ficoll, gelatin, and hydroxyethyl starch; antimicrobial preservatives such as benzalkonium chloride, benzethonium chloride, benzyl alcohol, chlorobutanol, m-cresol, myristyl gamma-picolinium chloride, paraben methyl, paraben propyl, phenol, 2-phenoxyethanol, phenyl mercuric nitrate, and thimerosal; chelating agents such as calcium disodium EDTA (ethylenediaminetetra acetic acid), disodium EDTA, calcium versetamide Na, calteridol, and DTPA; antioxidant and reducing agents such as acetone sodium bisulfate, argon, ascorbyl palmitate, ascorbate (sodium/acid), bisulfite sodium, butylated hydroxyl anisole, butylated hydroxyl toluene (BHT), cystein/cysteinateHCl, dithionite sodium, gentistic acid, gentistic acid ethanolamine, glutamate monosodium, glutathione, formaldehyde sulfoxylate sodium, metabisulfite potassium, metabisulfite sodium, methionine, monothioglycerol(thioglycerol), nitrogen, propyl gallate, sulfite sodium, tocopherol alpha, alpha tocopherol hydrogen succinate, thioglycolate sodium, thiourea, and anhydrous stannous chloride; solvents and co-solvents such as benzyl benzoate, oils, castor oil, cottonseed oil, N,N dimethylacetamide, ethanol, dehydrated ethanol, glycerin/glycerol, N-methyl-2-pyrrolidone, peanut oil, PEG, PEG 300, PEG 400, PEG 600, PEG 3350, PEG 4000, poppyseed oil, propylene glycol, safflower oil, sesame oil, soybean oil, vegetable oil, oleic acid, polyoxyethylene castor, sodium acetate-anhydrous, sodium carbonate-anhydrous, triethanolamine, and deoxycholate; buffers and pH-adjusting agents such as acetate, ammonium sulfate, ammonium hydroxide, arginine, aspartic acid, benzene sulfonic acid, benzoate sodium/acid, bicarbonate-sodium, boric acid/sodium, carbonate/sodium, carbon dioxide, citrate, diethanolamine, glucono delta lactone, glycine/glycine HCl, histidine/histidine HCl, hydrochloric acid, hydrobromic acid, lysine (L), maleic acid, meglumine, methanesulfonic acid, monoethanolamine, phosphate (acid, monobasic potassium, dibasic potassium, monobasic sodium, dibasic sodium and tribasic sodium), sodium hydroxide, succinate sodium/disodium, sulfuric acid, tartarate sodium/acid, and tromethamine (Tris); stabilizers such as aminoethyl sulfonic acid, asepsis sodium bicarbonate, L-cysteine, dietholamine, diethylenetriaminepentacetic acid, ferric chloride, albumin, hydrolyzed gelatin, insitol, and D,L-methionine; surfactants such as polyoxyethylene sorbitan monooleate (TWEEN® 80), Sorbitan monooleate, polyoxyethylene sorbitan monolaurate (TWEEN® 20), lecithin, polyoxyethylene-polyoxypropylene copolymers (PLURONICS®), polyoxyethylene monolaurate, phosphatidylcholines, glyceryl fatty acid esters, urea; complexing/dispersing agents such as cyclodextrins (e.g., hydroxypropyl-B-cyclodextrin, sulfobutylether-Bcyclodextrin); viscosity building agents such as sodium carboxymethyl cellulose, acacia, gelatin, methyl cellulose, polyvinyl and pyrrolidone.

In some embodiments, the one or more additives are inactive ingredients. Any suitable additive may be added to the cold solution, for example, any substance (at their indicated concentration ranges) on the FDA GRAS list, which is incorporated herein in its entirety. In some embodiments, the additives comprise one or more of a salt, a sugar, and a thickener. In an embodiment, the salt is NaCl at about 2.25% by mass or lower. In an embodiment, the sugar is glycerol at about 2% by mass or lower. In an embodiment, the thickener is CMC or Xanthan Gum at about 0.75% by mass or lower.

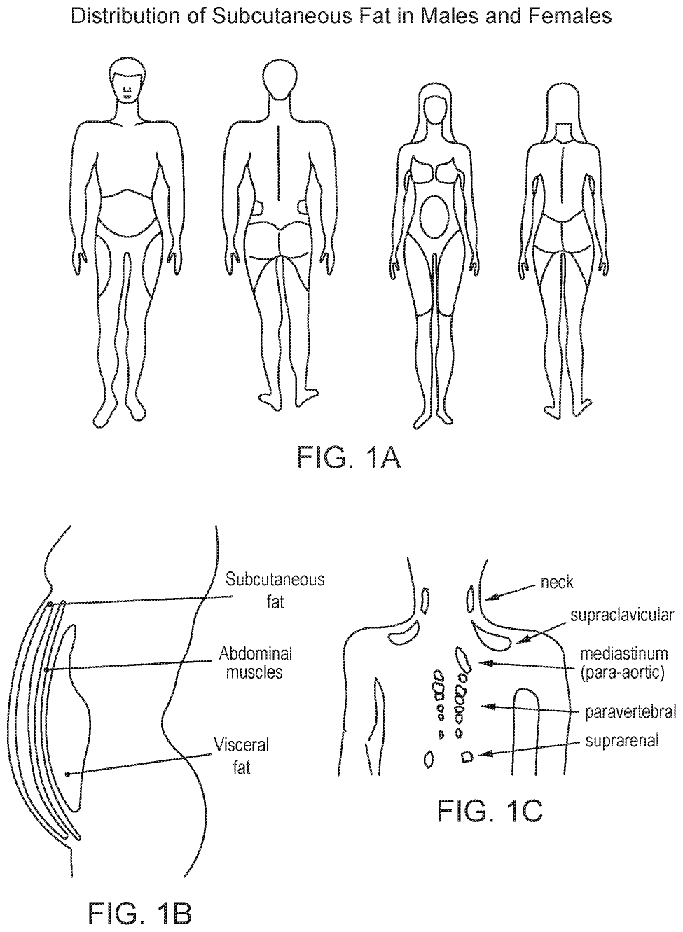

The cold solution can be delivered using a delivery device in accordance with the present invention to any fat tissue inside the body, including subcutaneous (including superficial and deep layers and sublayers and compartments therein), visceral, and brown adipose tissue. For example but not limiting, the cold solution can be delivered to fat tissue in any of the areas shown in FIGS. 1A-C, such as around the flank (i.e. “love handles”), abdomen, thigh area, upper arm, and submental area under the chin, and other areas as shown in the figures.

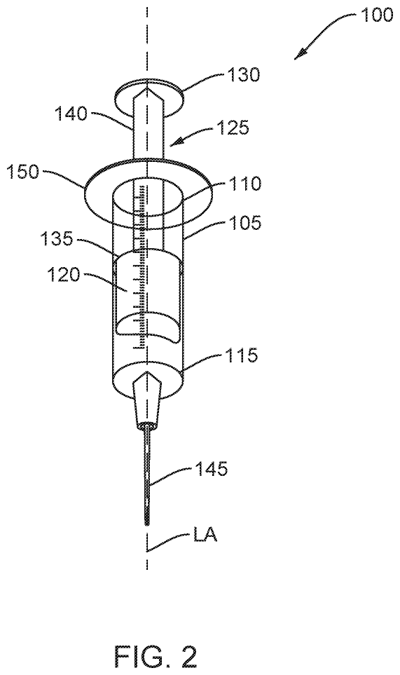

Any suitable delivery device may be used to deliver the cold solution to a subject. An exemplary device for delivering cold solution is generally shown in FIG. 2. The delivery device 100 includes a cylindrical member 105 having a first end 110 and a second end 115 along a longitudinal axis LA. The delivery device also includes an interior lumen 120 defined by the interior wall of the cylindrical member 105 and provided to receive and hold cold solution. The cylindrical member also includes a ledge 150, or “arms”, extending around the first end 110 out from the cylindrical member 105 along a plane that is orthogonal to the longitudinal axis LA. The ledge 150 also has an opening concentric with the interior lumen 120. The ledge helps facilitate handling and delivery of cold solution from the delivery device 100. In one embodiment, the delivery device 100 is a syringe-type device, for example, any suitable sterile syringe.

The cylindrical member 105 can be made of any type of biocompatible pharmacologically inert material suitable for use in holding and supplying fluids to be provided within a human body. Exemplary materials for the cylindrical member 105 include plastic, such as polyethylene or polypropylene, and glass. The delivery device 100 can be any size that suitable to hold one or more aliquots (doses) of cold solution for delivery to the desired tissue. The volume capacity of the delivery device 100 is typically between 1 ml and 60 ml, although capacity outside of those volumes is also contemplated.

The delivery device 100 also includes a plunger 125 at least partially disposed within the interior lumen 120. The plunger 125 is configured to move in and out of the cylindrical member 105 through the first end 110. The plunger 125 includes a head 130, a plunging member 135, and a rod 140 extending between the head 130 and plunging member 135 along the longitudinal axis LA. The plunging member 135 is disposed along the rod 140 at a predetermined distance from the head 130. The delivery device 100 also includes at least one needle 145 extending from the second end 115. The needle 145 typically has a thickness between 7 gauge and 34 gauge and a length between ¼ inch and 10 inches, such as about ¼ inch, ½ inch, 1 inch, 2 inches, 3 inches, 4 inches, 5 inches, 6 inches, 7 inches, 8 inches, 9 inches, or 10 inches. In one embodiment, the cylindrical member 105 narrows or tapers to a small opening at the second end 115, the small opening is configured to receive the needle 145. Preferably, the needle 145 is a hypodermic needle. Exemplary needle materials include, but are not limited to, stainless steel and carbon steel, with or without nickel plating.

The plunger 125, including the head 130 and the rod 140, can be any type of biocompatible, pharmacologically inert material suitable for coming in contact with fluids to be provided within a human body. Exemplary materials for the plunger 125 include plastic, such as polyethylene or polypropylene, and glass. With respect to the plunging member, a portion or all of the plunging member 135 can be a rubber material, such that a seal is formed between the sides of the plunging member 135 and the interior wall of the cylindrical member 105. The rubber material can be any rubber suitable for coming in contact with fluids to be provided to the human body, such as natural rubber latex or a synthetic rubber. In some embodiments, the delivery device 100 can also include an agitator (not shown) disposed within the interior lumen 120 configured to mix the cold solution ingredients.

Once the cold solution is ready for delivery to tissue using the delivery device 100, the needle 145 is used to pierce the skin. Once the needle 145 is through the skin and positioned at or near the target tissue, the plunger 125 is forced downward toward the second end 115 of the cylindrical member 105. The force of the plunging member 135 on the cold solution forces the cold solution through the cylindrical member 105, out the needle 145, and into (or near) the target tissue. In one embodiment, more than one needle is provided at the second end 115 of the delivery device 100. The more than one needle can be provided in single row array, multiple row array, circular pattern, or any other conceivable arrangement.

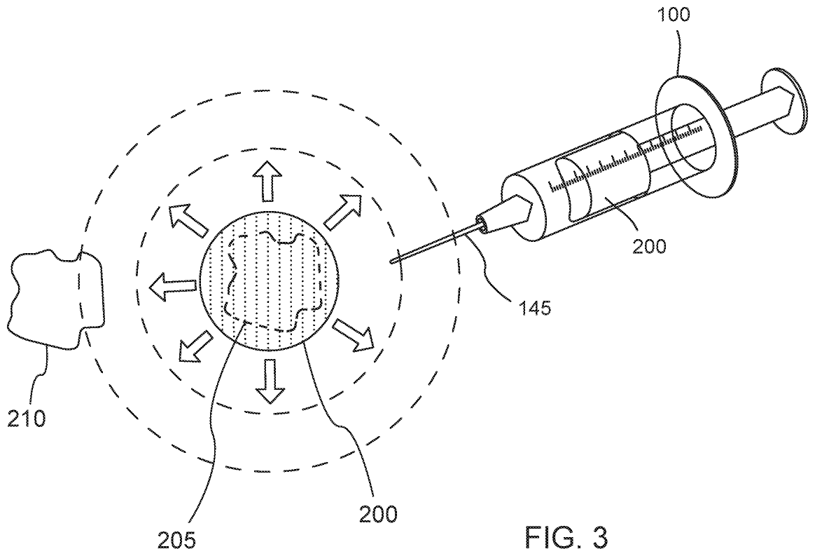

In a preferred embodiment, the cold solution is delivered to or adjacent to adipose tissue (fat tissue) within a subject's body in order to induce apoptosis of the tissue cells and reduce the tissue, as shown generally in FIG. 3. Using the cold solution to reduce fat can improve the subject's appearance. Referring to the procedure of FIG. 3, the device 100 of FIG. 2 is used to deliver cold solution 200 to adipose tissue 205. (In other examples, cold solution can be delivered using a syringe-type device, a catheter or a cannula.) The needle 145 is inserted through the subject's skin and advanced to a location at or near the target adipose tissue 205 (shown in phantom line). The cold solution 200 is then delivered and cools the adipose tissue 205.

After delivery, an area affected by the cold solution 200 expands to a size larger than the initial delivery site (shown in the figure as arrows radiating outwardly from the delivered cold solution 200 and dashed circles of increasing size). The cooling effect of the cold solution 200 is localized to the adipose tissue 205 and possibly surrounding tissue, such as adjacent tissue 210. In this way, discomfort caused by the cold treatment is limited. The cold solution 200 is sterile and biocompatible; and, as such, the cold solution 200 can be advantageously left in the body (e.g. no removal of the cold solution is necessary after cooling has been effected).

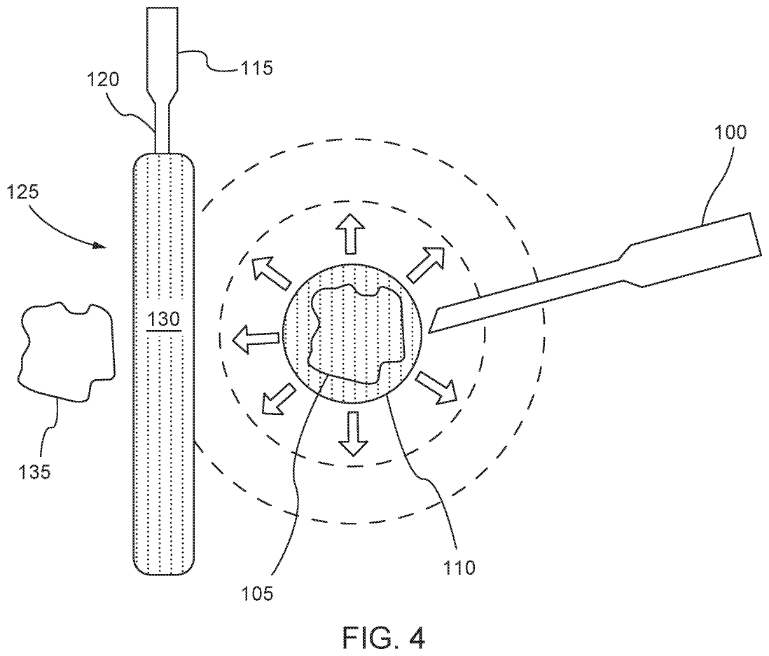

In some embodiments, a cold solution containment device can be used in combination with delivery device 100, for example, a device comprising a balloon configured for controlling the cooling effect of the cold solution, as shown in FIG. 4. A deployment device 115 having an application cannula 120 is inserted through the patient's skin. At the distal end of the application cannula 120, there is a controlling end 125. The deployment device 115 is advanced until the controlling end 125 is at a location between the target tissue 105 and an adjacent (surrounding) tissue 135. The controlling end 125 includes a balloon 130. While the balloon 130 is shown having a linear shape, it can have any shape, such as a ring that encircles the target tissue 105. In some embodiments, the balloon 130 is filled with air to create a barrier between the adjacent tissue 135 and the spreading cold solution 110. The balloon 130 limits heat transferring from the adjacent tissue 135 to the cold solution 110. In some embodiments, the delivery device 100 comprises a cannula such as a needle. In some embodiments, the containment device is the delivery device, for example, balloon 130 can be filled with cold solution so as to deliver and contain the solution to a particular area.

In an exemplary procedure, a practitioner identifies which adipose tissue on a subject's body is the target for cold solution treatment. The area of the subject's skin overlying the target tissue adipose is cleaned and an entry point is marked on the skin through which a device for delivering cold solution will enter. The entry point can be identified visually, or through the use of one or more imaging technique such as ultrasound, magnetic resonance and x-ray. The device is then inserted into the entry point and advanced to the target tissue. The cold solution is then injected at (or near) the target tissue. An amount of cold solution can be delivered to multiple sites at (or near) the target tissue. In some instances, injection to multiple sites increases the amount of target tissue that is exposed to the cold solution and cooled and can improve the effectiveness of the treatment. The solution can be delivered using one or more injection patterns, for example, one or more boluses, a plow, fan, or grid-like pattern, or other injection techniques known to those of skill in the art. Optionally, a massaging step post injection may be utilized to increase fat cell damage.

In an embodiment of the present invention, a treatment plan can be created for a subject, for example to determine the solution properties, volume of solution to be delivered, and treatment sites. Factors considered in creating a treatment plan for a subject may comprise one or more of gender, height, body weight, body fat percentage, anatomy, lifestyle, vitals, medical history, lipid profiles, skin elasticity, medication, nutrition, supplements, demographic, fat saturation, and the like. Fat saturation may be characterized by one or more of imaging, biopsy, and impedance measurement. In embodiments of the present invention, once a plan is created for the subject, the amount of solution to the administered can be adjusted based on one or more of the area or areas to be treated, the depth of injection, and the injection pattern to be used.

A computer or artificial intelligence system may be utilized to create a treatment plan for a patient by collecting pre-, peri-, and/or post-injection data from multiple subjects. It is appreciated that the more data points, the more effective the artificial intelligence system will be in creating a treatment plan for a subject. For example, pre-, peri-, and/or post-injection data may be collected for each subject comprising one or more of gender, height, body weight, body fat percentage, the subject's anatomy, lifestyle, the subject's vitals, medical history, lipid profiles, skin elasticity, medication, nutrition, supplements, demographic, fat saturation, imaging data, treatment data and fat loss data. Data may be measured by any suitable means. For example, fat loss data may be measured by calipers or any imaging methods such as ultrasound and/or MRI.

Areas in which the cold solution can be delivered to fat tissue include, without limitation, the face, neck, submental area under chin, jowls, eyelids, posterior neck (buffalo hump), back, shoulders, arms, triceps, biceps, forearms, hands, chest, breasts, abdomen, abdominal etching and sculpting, flanks (love handles), lower back, buttocks (banana roll), hips (saddle bags), anterior and posterior thighs, inner thighs, mons pubis, vulva, knees, calves, shin, pretibial area, ankles and feet.

The aforementioned procedures are also useful for treating obesity and weight-related disorders. Generally, treatment methods include administering an effective amount of cold solution (as described above) to a subject in need of treatment, including a subject that has been diagnosed to be in need of such treatment.

The treatment methods can include identifying a subject in need of treatment (e.g., a subject having, or at risk of having, obesity or developing a weight-related disorder), and administering to the subject an effective amount of cold solution (as described above). In a convenient example, the subject is diagnosed as being an overweight or obese subject (e.g., having a body mass index (BMI) of 25-29 or 30 or above) or a subject with a weight-related disorder. A subject in need of treatment can be selected based on the subject's body weight or BMI.

In some examples of the treatment method, subject selection can include assessing the amount adipose tissue in the subject and recording these observations. The evaluation can be performed before, during, and/or after the delivery of cold solution. For example, the evaluation can be performed at least 1 day, 2 days, 4, 7, 14, 21, 30 or more days before and/or after the delivery of cold solution.

The treatment methods can include assessing the treatment. For example, the amount of adipose tissue in the subject following treatment is observed and recorded. This post-treatment observation can be compared to the observations made during subject selection. In some instances, the subject will have decreased amount of adipose tissue. In other instances, the subject will show reduced symptoms.

The treatment assessment can include determining the subject's weight or BMI before and/or after treatment, and comparing the subject's weight or BMI before treatment to the weight or BMI after treatment. An indication of success would be an observation of a decrease in weight or BMI. In some examples, the treatment is administered one or more additional times until a target weight or BMI is achieved. Alternatively, measurements of girth can be used, e.g., waist, chest, hip, thigh, or arm circumference.

The treatment assessment can be used to determine the future course of treatment for the subject. For example, treatment may be continued without change, continued with change (e.g., additional treatment or more aggressive treatment such as an increase in volume delivered or a cold solution comprising different ingredients), or treatment can be stopped. The treatment methods can include one or more additional deliveries of cold solution, e.g., to further reduce the amount of adipose tissue to maintain or further reduce obesity in the subject.

In another aspect of the invention, the cold solution and methods described above can be provided to a tissue within the body of a patient, for example, for the treatment of a patient. The tissue to which the cold solution can be administered includes one or more of connective, epithelial, neural, joint, cardiac, hepatic, renal, vascular, cutaneous, and muscle tissue. Additionally, methods include delivery of a cold solution to any one or more of the following locations: proximate to a nerve, proximate to subcutaneous adipose tissue, proximate to breast tissue, proximate to visceral fat, fatty tissue proximate to the pharynx, fatty tissue proximate to the palate, fatty tissue proximate to the tongue, proximate to a spinal cord lipoma, proximate to visceral fat, proximate to lipomastia, proximate to a tumor, proximate to cardiac tissue, proximate to pericardial fat, proximate to epicardial fat, proximate to a lipid-rich plaque in the vasculature, and proximate to areas of steatosis or ectopic fat in muscle. Various conditions, disorders, or diseases which can be treated through delivery of cold solution to a subject include obesity, sleep apnea, lipedema, lymphedema, non-alcoholic steatohepatitis, atrial fibrillation, atherosclerosis, and nerve pain.

### EQUIVALENTS

While the present invention has been described in conjunction with certain preferred embodiments, one of ordinary skill, after reading the foregoing specification, will be able to effect various changes, substitutions of equivalents, and other alterations to the apparatuses and methods set forth herein.

1. A method for reducing adipose tissue in a subject, the method comprising:
delivering a cold solution having no ice particles from a delivery device to adipose tissue underneath a subject's skin thereby cooling the adipose tissue.

2. The method of claim 1, wherein the cold solution comprises water.

3. The method of claim 2, further comprising at least one salt.

4. The method of claim 3, wherein the at least one salt is sodium chloride, potassium chloride, calcium chloride, magnesium chloride, disodium hydrogen phosphate, sodium phosphate, potassium phosphate, calcium phosphate, magnesium phosphate, sodium carbonate, potassium carbonate, calcium carbonate, magnesium carbonate, sodium lactate, potassium lactate, calcium lactate, magnesium lactate, or a combination thereof.

5. The method of claim 3, wherein the cold solution comprises the at least one salt at about 2.25% or lower.

6. The method of claim 2, further comprising at least one sugar.

7. The method of claim 6, wherein the at least one sugar is glycerol, glucose, mannitol, hetastarch, sucrose, sorbitol or a combination thereof.

8. The method of claim 6, wherein the cold solution comprises the at least one sugar at about 2% or lower.

9. The method of claim 2, further comprising at least one thickener.

10. The method of claim 9, wherein the at least one thickener comprises sodium carboxymethylcellulose (CMC), xanthan gum or a combination thereof.

11. The method of claim 9, wherein the cold solution comprises the at least one thickener at about 0.75% or lower.

12. The method of claim 1, wherein the cold solution comprises at least one ion, polysaccharide, lipid, oil, lysolecithin, amino acid, caffeine, surfactant, antimetabolite, detergent or a combination thereof.

13. The method of claim 12, wherein the at least one ion is calcium, potassium, hydrogen, chloride, magnesium, sodium, lactate, phosphate, zinc, sulfur, nitrate, ammonium, carbonate, hydroxide, iron, barium, salts thereof or a combination thereof.

14. The method of claim 12, wherein the at least one oil is canola oil, coconut oil, corn oil, cottonseed oil, flaxseed oil, olive oil, palm oil, peanut oil, safflower oil, soybean oil, sunflower oil or a combination thereof.

15. The method of claim 12, wherein the surfactant is a detergent.

16. The method of claim 15, wherein the detergent is at least one of deoxycholate, sodium tetradecyl sulphate, polidocanol, deoxycholate, sodium tetradecyl sulphate, polidocanol, polysorbate 20 (polyoxyethylen (20) sorbitan monolaurate), polysorbate 40 (polyoxyethylene (20) sorbitan monopalmitate), polysorbate 60 (polyoxyethylene (20) sorbitan monostearate), polysorbate 80 (polyoxyethylene (20) sorbitan monooleate), sorbitan ester, poloxamater or a combination thereof.

17. The method of claim 1, wherein the cold solution comprises one or more of water, a salt, a sugar and a thickener.

18. The method of claim 1, wherein the cold solution has a temperature comprising about +10° C. to about −50° C.

19. The method of claim 1, wherein the cold solution is cooled.

20. The method of claim 1, wherein the cold solution is supercooled.

21. The method of claim 1, wherein delivering the cold solution includes injecting the cold solution using a cannula.

22. The method of claim 21, wherein the cannula further comprises a balloon.

23. The method of claim 1, wherein delivering the cold solution includes delivering the cold solution through a catheter.

24. The method of claim 1, wherein the adipose tissue is any one of: subcutaneous, visceral, and brown adipose tissue.

25. The method of claim 1, wherein the cold solution is delivered to adipose tissue in one or more areas selected from the group consisting of: tissue around the flank, abdomen, thigh area, upper arm, and submental area under the chin.

26. The method of claim 1, wherein delivering the cold solution includes delivering the cold solution to tissue adjacent to the adipose tissue.

27. The method of claim 1 further comprises administrating an effective amount of cold solution to treat obesity or a weight-related disorder.

28. The method of claim 27 further comprising selecting the subject to whom to administer the cold solution.

29. The method of claim 28 further comprising creating a treatment plan for the subject.

30. The method of claim 29 further comprising assessing the result of administrating the cold solution.

31. A cold solution for reducing adipose tissue in a subject, comprising:
water having no ice particles configured to be delivered to adipose tissue underneath a subject's skin thereby cooling the adipose tissue.

32. The solution of claim 31, further comprising at least one salt.

33. The solution of claim 32, wherein the at least one salt is sodium chloride, potassium chloride, calcium chloride, magnesium chloride, disodium hydrogen phosphate, sodium phosphate, potassium phosphate, calcium phosphate, magnesium phosphate, hydrogen, sodium carbonate, potassium carbonate, calcium carbonate, magnesium carbonate, sodium lactate, potassium lactate, calcium lactate, magnesium lactate, or a combination thereof.

34. The solution of claim 32, wherein the cold solution comprises the at least one salt at about 2.25% or lower.

35. The solution of claim 31, further comprising at least one sugar.

36. The solution of claim 35, wherein the at least one sugar is glycerol, glucose, mannitol, hetastarch, sucrose, sorbitol or a combination thereof.

37. The solution of claim 35, wherein the cold solution comprises the at least one sugar at about 2% or lower.

38. The solution of claim 31, further comprising at least one thickener.

39. The solution of claim 38, wherein the at least one thickener comprises sodium carboxymethylcellulose (CMC), xanthan gum or a combination thereof.

40. The solution of claim 38, wherein the cold solution comprises the at least one thickener at about 0.75% or lower.

41. The solution of claim 31, wherein the cold solution comprises at least one ion, polysaccharide, lipid, oil, lysolecithin, amino acid, caffeine, surfactant, antimetabolite, detergent or a combination thereof.

42. The solution of claim 41, wherein the at least one ion is calcium, potassium, hydrogen, chloride, magnesium, sodium, lactate, phosphate, zinc, sulfur, nitrate, ammonium, carbonate, hydroxide, iron, barium, salts thereof or a combination thereof.

43. The solution of claim 41, wherein the at least one oil is canola oil, coconut oil, corn oil, cottonseed oil, flaxseed oil, olive oil, palm oil, peanut oil, safflower oil, soybean oil, sunflower oil or a combination thereof.

44. The solution of claim 41, wherein the surfactant is a detergent.

45. The solution of claim 44, wherein the detergent is at least one of deoxycholate, sodium tetradecyl sulphate, polidocanol, deoxycholate, sodium tetradecyl sulphate, polidocanol, polysorbate 20 (polyoxyethylen (20) sorbitan monolaurate), polysorbate 40 (polyoxyethylene (20) sorbitan monopalmitate), polysorbate 60 (polyoxyethylene (20) sorbitan monostearate), polysorbate 80 (polyoxyethylene (20) sorbitan monooleate), sorbitan ester, poloxamater or a combination thereof.

46. The solution of claim 31, wherein the cold solution comprises one or more of water, a salt, a sugar and a thickener.

47. The solution of claim 31, wherein the cold solution has a temperature comprising about +10° C. to about −50° C.

48. The solution of claim 31, wherein the cold solution is cooled.

49. The solution of claim 31, wherein the cold solution is supercooled.

50. The solution of claim 31, wherein the cold solution is injected using a cannula.

51. The solution of claim 50, wherein the cannula further comprises a balloon.

52. The solution of claim 31, wherein the cold solution is delivered through a catheter.

53. The solution of claim 31, wherein the adipose tissue is any one of: subcutaneous, visceral, and brown adipose tissue.

54. The solution of claim 31, wherein the cold solution is delivered to adipose tissue in one or more areas selected from the group consisting of: tissue around the flank, abdomen, thigh area, upper arm, and submental area under the chin.

55. The solution of claim 31, wherein the cold solution is delivered to tissue adjacent to the adipose tissue.

56. The solution of claim 31 wherein the cold solution is delivered to treat obesity or a weight-related disorder.
